(19) United States
(12) Patent Application Publication
(10) Pub. No.: US 2012/0045831 A1
(43) Pub. Date: Feb. 23, 2012

(54) POLY(LACTIC-GLYCOLIC)ACID CROSS LINKED ALENDRONATE (PLGA-ALN) A SHORT TERM CONTROLLED RELEASE SYSTEM FOR STEM CELL DIFFERENTIATION AND DRUG DELIVERY

(75) Inventors: Mei-Ling Ho, Kaohsiung City (TW); Je-Ken Chang, Kaohsiung City (TW); Rajalakshmanan Eswaramoorthy, Kaohsiung City (TW); Shun-Cheng Wu, Kaohsiung City (TW)

(73) Assignee: KAOHSIUNG MEDICAL UNIVERSITY, Kaohsiung City (TW)

(21) Appl. No.: 12/860,377

(22) Filed: Aug. 20, 2010

(51) Int. Cl. C12N 5/00; C08G 63/06

(52) U.S. Cl. 435/377; 528/361

(57) ABSTRACT

The present invention relates to a bisphosphonates and synthetic polymeric carriers to form a sustained release system. The present invention also provides a method for preparing a sustained release system, comprising activation of the synthetic polymeric carriers and the cross linking reaction. The present invention further provides methods for enhancing stem cell differentiation into osteogenic lineage and chondrogenic lineage, which comprise culturing a population of stem cells in specific micro-environments.

### FIELD OF THE INVENTION

The present invention relates to a short term controlled release composition and a method for preparing the composition thereof. More specifically the invention relates to a composition which is applicable to the technical field of stem cell based tissue engineering.

### BACKGROUND OF THE INVENTION

The induction factors play a major role in directing stem cells differentiation into tissue specific cells, and thus they can be applied in tissue engineering (Lutolf and Hubbell, Nat Biotechnol, 2005, 23:47-55). Induction factors can be either protein-based or chemical-based (Gaissmaier et al, Injury, 2008, Suppl 1: S88-96; Zur Nieden et al, BMC Dev Biol, 2005, 5:1); however, these induction factors have their drawbacks including expensive, may damage tissues, or difficult to deliver. Therefore, it is important to search new induction factors that can initiate and/or facilitate the differentiation of stem cells thus promote subsequent specific matrices deposition resulting in regeneration in vivo. It has been reported that bone morphogenetic protein-2 (BMP-2) plays an important role in the early stage of differentiation process of adult stem cells into osteoblasts or chondrocytes (Chen et al, Growth Factors, 2004, 22:233-241; Shea et al, J Cell Biochem, 2003, 90:1112-1127; Kato et al, Life Sci, 2009, 84:302-310). Previous reports also showed that BMP-2 induces mesenchymal stem cells differentiation and promotes bone and cartilage repair in-vitro and in-vivo (Gaissmaier et al, Injury, 2008, Suppl 1: S88-96; Zhao et al, J Control Release, 2010, 141:30-37; Diekman et al, Tissue Eng Part A, 2009; Mrugala et al, Cloning Stem Cells, 2009, 11:61-76; Park et al, J Biosci Bioeng, 2009, 108:530-537 Hou et al, Biotechnol Lett, 2009, 31:1183-1189).

Bisphosphonates are the commonly used drugs to treat osteoporosis (Russell, Pediatrics, 2007, 119 Suppl 2:S150-162; Rogers, Curr Pharm, 2003, 9:2643-2658; Fisher et al, Endocrinology, 2000, 141:4793-4796). Alendronate is one of the bisphosphonates acts through interferes the mevalonate pathway in osteoclasts. Recent reports also indicated that alendronate stimulates the mesenchymal stem cells (MSCs) to differentiate into osteogenic lineage. Recently, our studies exploring the effects of short term treatment of bisphosphonates on human bone marrow mesenchymal stem cells (BMSCs) and adipose derived stem cells (ADSCs) increases the BMP-2 expression in a time dependent manner in alendronate treated cultures. We also found that bisphosphonate enhanced the microenvironment induced differentiation of MSCs into different lineages. We used a short term treatment of alendronate on human adipose derived stem cells (hADSCs) implanted in rat calvarial defect showed the better bone repair. We also found that ALN enhanced the HA microenvironment induced chondrogenesis in hADSCs cultured under HA coated dishes. However, better short term controlled releasing carrier for alendronate in stem cell based tissue engineering has still ongoing debate (Cartmell, J Pharm Sci, 2009, 98:430-441).

### BRIEF DESCRIPTION OF THE DRAWINGS

The invention can be more fully understood by reading the subsequent detailed description and examples with references made to the accompanying drawing, wherein:

FIG. 1. Scanning electron microscopy (SEM) image of (a) PLGA, (b) PLGA-ALN scaffold, (c & d) PLGA-ALN microspheres.

FIG. 2. Cell viability by 3-(4,5-dimethylthiazol-2-yl)-2,5-diphenyltetrazolium bromide (MTS) analysis

FIG. 3. Releasing profile for Alendronate from PLGA-ALN carriers

FIG. 4. (A) Alizarin red S staining for mineralization and (B) quantification of mineralization in PLGA-ALN-M cultured hADSCs

FIG. 5. RT-PCR analysis for osteogenic gene expressions in PLGA-ALN-M cultured hADSCs

FIG. 6. Treatment of PLGA-ALN-M enhanced chondrogenesis through the aggregation of hADSCs cultured under HA microenvironment for after 2 hrs.

FIG. 7. Chondrogenic gene expressions in PLGA-ALN-M treated hADSCs.

FIG. 8. Radiographic images of hADSCs seeded (a) PLGA and (b) PLGA-ALN-3D treated rat calvarial defect after 8 weeks.

FIG. 9. Micro CT analysis of hADSCs seeded (a) PLGA and (b) PLGA-ALN-3D treated rat calvarial defect after 8 weeks.

### SUMMARY OF THE INVENTION

The present invention provides a short term controlled released composition which comprises poly(lactic-co-glycolic acid) (PLGA) cross-linked alendronate (ALN), wherein is constructed into 3D scaffold (PLGA-ALN-3D) or microsphere (PLGA-ALN-M). The present invention also provides a method for preparing a short term controlled release composition, which comprises activating a carboxylic acid end group of PLGA to produce ethyl(dimethylaminopropyl) carbodiimide (EDC)/N-Hydroxysuccinimide (NHS) activated PLGA; and performing cross linking reaction between EDC/NHS activated PLGA and sodium alendronate. The present invention further provides a method for enhancing stem cell differentiation into osteogenic lineage, which comprises culturing stem cells in micro-environment with PLGA-ALN. The present invention also provides a method for enhancing stem cell differentiation into chondrogenic lineage, which comprises culturing a population of stem cells in micro-environment with hyaluronan (HA) and PLGA-ALN.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

Natural and synthetic polymeric carriers (micro- and nano-spheres) have been developed as an effective method to control the release of the drugs (Cartmell, J Pharm Sci, 2009, 98:430-441; Bhardwaj et al, J Diabetes Sci Technol, 2008, 2:1016-1029; Mundargi et al, J Control Release, 2008, 125:193-209). The excellent biocompatibility and biodegradability makes, poly(lactic-co-glycolic acid) (PLGA) and poly(lactic acid) (PLA) were more appropriate carriers for the application of drug delivery (Lim et al, J Mater Sci Mater Med, 2009, 20:1669-1675). Recently, we reported that PLGA modified HA scaffolds showed the better chondrogenic effect on hADSCs (Wu et al, Biomaterials, 2010, 31:631-640). Therefore, we hypothesized that PLGA cross linked alendronate may be the better carrier for short term slow release of alendronate and it has the potential to enhance the differentiation of human adipose derived stem cells (hADSCs).

One embodiment of the present invention provides a short term controlled release composition which comprises poly(lactic-co-glycolic acid) (PLGA) cross-linked alendronate (ALN), which is constructed into 3D scaffolds (PLGA-ALN-3D) or microspheres (PLGA-ALN-M).

The PLGA-ALN-3D scaffolds of the short term controlled release composition have pores size of 150-300 μm and average porosity of 85%.

The PLGA-ALN-M microspheres of the short term controlled release composition are 50-100 μm in diameter with smooth surface.

The present invention further provides a method for preparing a short term controlled release composition, which comprises the following steps: (a) activating a carboxylic acid end group of PLGA to produce ethyl(dimethylaminopropyl) carbodiimide (EDC)/N-Hydroxysuccinimide (NHS) activated PLGA; and (b) performing cross linking reaction between EDC/NHS activated PLGA and sodium alendronate.

The carboxylic acid end group of PLGA of the method for preparing a short term controlled release composition is activated by ethyl(dimethylaminopropyl) carbodiimide (EDC)/N-Hydroxysuccinimide (NHS) method.

The EDC/NHS method of the method for preparing a short term controlled release composition further comprises mixing NHS, EDC and PLGA. NHS and EDC are mixed in ratio 3:2. PLGA is dissolved in dichloromethane. The carboxylic acid end group activated PLGA of the EDC/NHS method is precipitated by excess diethyl ether.

The cross-linking reaction of the method for preparing a short term controlled release composition further comprising reacting EDC/NHS activated PLGA and sodium alendronate in the same mole ratio. The cross linking reaction is performed in dry dimethysulphoxide.

One embodiment of the present invention also provides a method for enhancing stem cell differentiation into osteogenic lineage, which comprises culturing stem cells in micro-environment with PLGA-ALN.

The PLGA-ALN of the method for enhancing stem cell differentiation into osteogenic lineage is constructed into 3D scaffolds (PLGA-ALN-3D) or microspheres (PLGA-ALN-M). The PLGA-ALN-3D scaffolds have the pores size of 150-300 μm and average porosity of 85%. The PLGA-ALN-M microspheres are 50-100 μm in diameter with smooth surface.

The stem cells of the method for enhancing stem cell differentiation into osteogenic lineage are adipose derived stem cells (ADSCs) of human origin.

The present invention also provides a method for enhancing stem cell differentiation into chondrogenic lineage, which comprises culturing a population of stem cells in micro-environment with hyaluronan (HA) and PLGA-ALN.

The PLGA-ALN of the method for enhancing stem cell differentiation in to chondrogenic lineage is constructed into microspheres (PLGA-ALN-M). The PLGA-ALN-M microspheres are 50-100 μm in diameter with smooth surface. The stem cells of the method for enhancing stem cell differentiation into chondrogenic lineage are adipose derived stem cells (ADSCs) of human origin.

PLGA cross linked ALN enhanced the osteogenic or chondrogenic differentiation under the osteo-induction condition or chondro-induction condition respectively, induced hADSCs. And the cross linking between PLGA and ALN do not affect the efficiency of ALN. Therefore, PLGA-ALN is a short term controlled release carrier for enhancing osteogenic and chondrogenic differentiation in committed hADSCs for the regeneration of bone and cartilage leads to the application in stem cell based tissue engineering.

### EXAMPLES

The examples below are non-limiting and are merely representative of various aspects and features of the present invention.

### Example 1

### Isolation and Culture of hADSCs

After obtaining informed consent from all the patients and approval from the Kaohsiung Medical university hospital ethics committee, leftover subcutaneous adipose tissue was acquired from patients undergoing orthopedic surgery. The hADSCs were isolated from human subcutaneous adipose tissue following the previously described method (Fehrer and Lepperdinger, Exp Gerontol, 2005, 40:926-930). The isolated hADSCs were cultured and expanded at 37 C under 5% CO₂ in K-NAC medium containing Keratinocyte-SFM (Gibco BRL, Rockville, Md.) supplemented with the EGF-BPE (Gibco BRL, Rockville, Md.), N-acetyl-L-cysteine, L-ascorbic acid 2-phosphate sequimagnesium salt (Sigma, St. Louis, Mo.) and 5% FBS (Fehrer and Lepperdinger, Exp Gerontol, 2005, 40:926-930).

### Example 2

### Synthesis of PLGA Cross Linked Alendronate (PLGA-ALN)

The fabrication of PLGA-ALN is the two stage process, first the activation of the carboxylic acid end group of PLGA by EDC/NHS method and second is the cross linking reaction. Briefly, 1 g of PLGA (50/50) dissolved in 10 ml of dichloromethane was reacted with 3:2 ratio of NHS and EDC, stirred at room temperature for 12 h. Then, the insoluble dicyclohexylurea was removed by using a 0.45 μm Teflon filter. The activated PLGA polymer product was precipitated by excess diethyl ether, followed by dried under vacuum for 4 h. The PLGA-ALN was prepared by reacting equivalent mole ratio of EDC/NHS activated PLGA with sodium alendronate in dry dimethylsulphoxide stirred under room temperature for 12 h. The final product was precipitated and isolated by adding excess of cold diethyl ether followed by double distilled water. The isolated PLGA-ALN was dried under vacuum and stored at −20 C till use.

### Example 3

### Fabrication of Porous PLGA-ALN-3D Scaffolds

The porous scaffolds for the PLGA-ALN were prepared by the salt leaching method. Briefly, 1:6 weight ratios of PLGA-ALN and combined with NaCl salt (particle size was 300-400 μm) was dissolved in 10 mL of chloroform under magnetic stirring. The gel-like precipitate was mixed completely with sieved salt particulates and was put into 2-mm thick, 5-mm in diameter disc-shaped Teflon molds, followed by a partial evaporation of chloroform at room temperature to obtain a semi-solidified mass. The molds were then immersed in a distilled water solution at room temperature, as well as salt leaching within the polymer/salt matrices. Then the porous polymeric scaffolds were taken out from the molds, washed with distilled water three times, and then dried under vacuum for 1 day.

### Example 4

### PLGA-ALN-M Microsphere Preparation and Characterization

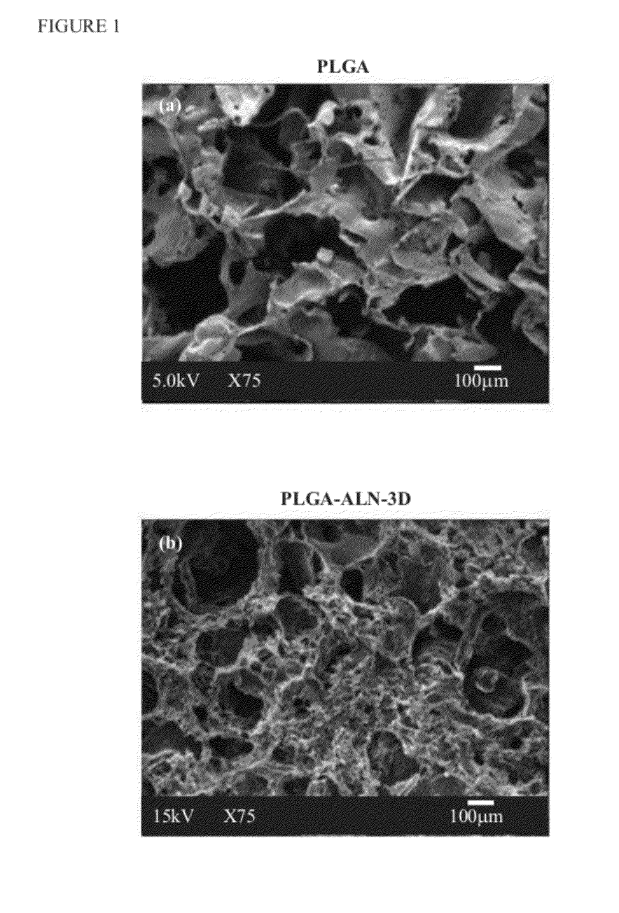

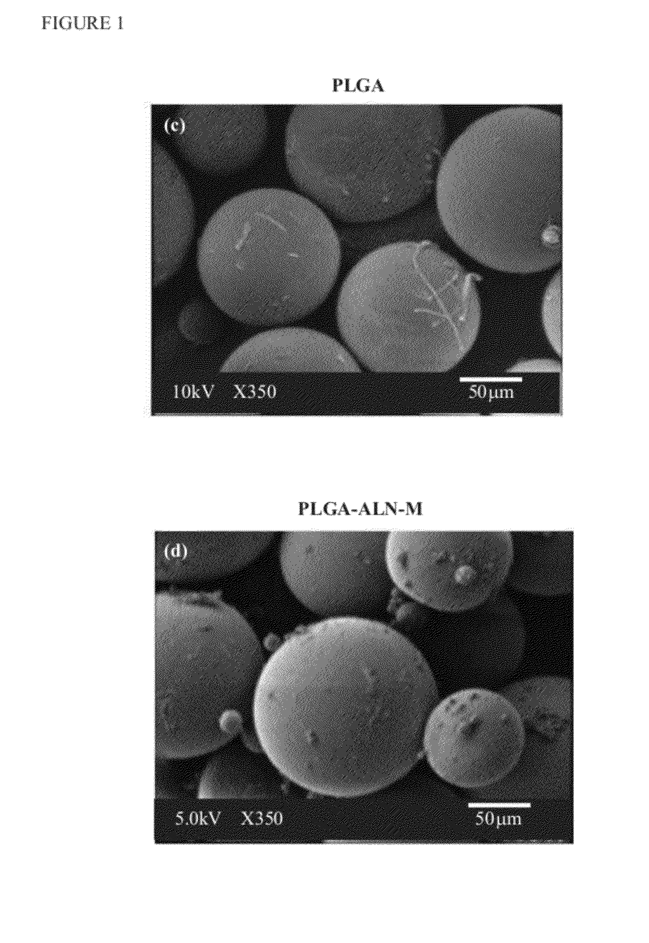

The microspheres were fabricated by the o/w emulsion technique (FIG. 1). Briefly, 10% PLGA-ALN polymer solution was prepared by dissolved in dichloromethane (DCM). The single emulsion (o/w) was formed by gradual addition of the polymer solution into the 20 ml of 1% aqueous PVA solution under vigorous stirring. The solution was stirred at room temperature for 30 mins to harden the microspheres, followed by the dichloromethane was evaporated under water suction and then centrifuged to collect solid microspheres. The resultant microspheres were washed with distilled water three times and freeze dried. The overall morphology of the microspheres was examined using scanning electron microscopy (SEM) (Hitachi S3200, Tokyo, Japan) after gold coating of the microsphere samples on a stub and the mean size of the microspheres were measured by particle size analyzer.

### Example 5

### Release Kinetics

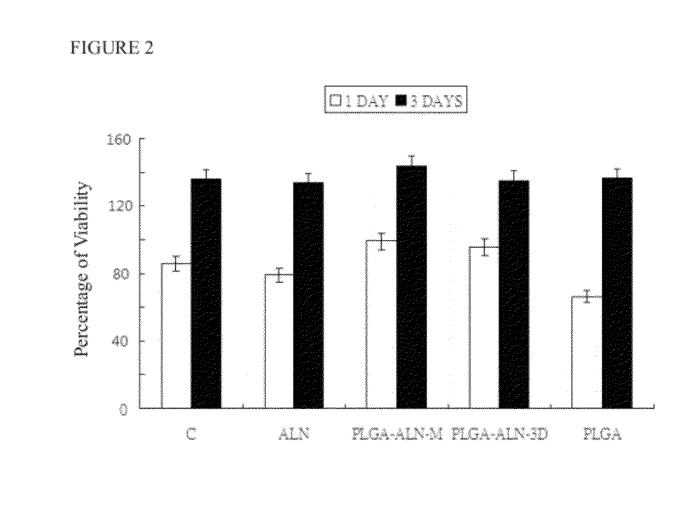

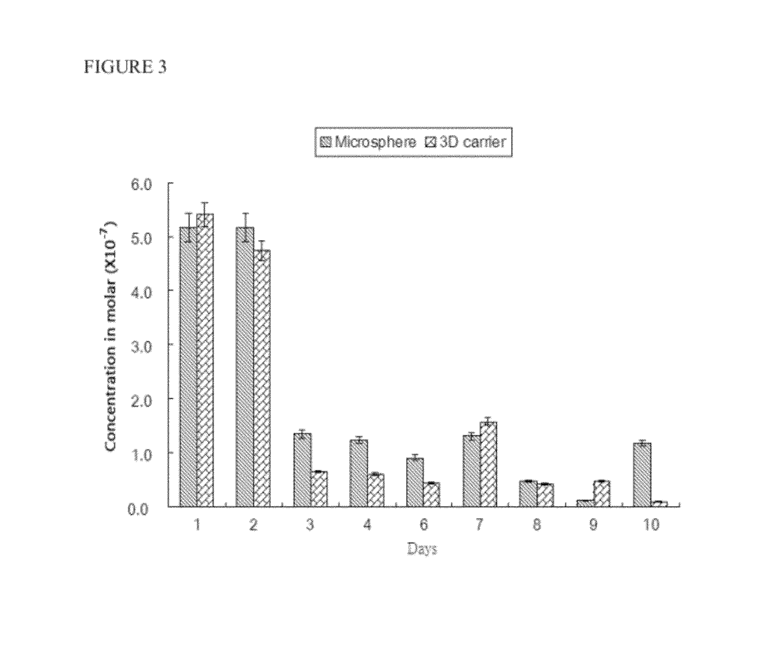

Ten-milligram PLGA-ALN-M or PLGA-ALN-3D scaffolds were suspended in 1 mL of PBS and the 500 uL of solution was collected from the mixture and replaced with fresh PBS. The concentration of the released alendronate was measured by reported spectrophotometric method. Results from release kinetics studies data showed that PLGA-ALN-3D and PLGA-ALN-M were released the effective concentration in the range of 5×10⁻⁷M to 5×10⁻⁸M of alendronate for 9 days (with daily average concentration of 1×10⁻⁷M) (FIG. 3).

### Example 6

### Scanning Electron Microscopy (SEM) Examination

The morphological characteristics of PLGA-ALN scaffolds were observed by using scanning electron microscopy (SEM, JEOL, Tokyo, Japan). However, samples were first coated with gold via a sputter-coater at ambient temperature. Micrographs of both scaffolds were taken at 50× and 100×. The overall morphology of the scaffolds was examined after gold coating of the scaffold samples on a stub and the mean pores size of the scaffolds were 150-300 μm, with average porosity of 85% (FIG. 1a-b). The PLGA-ALN-M was 50-100 μm in diameter with smooth surface (FIG. 1c-d).

### Example 7

### Cell Culture in PLGA-3D and PLGA-ALN-3D Scaffold

Cells/scaffold constructs of PLGA-3D and PLGA-ALN-3D scaffolds with hADSCs were prepared. The PLGA-3D and PLGA-ALN-3D scaffolds were pre-wetted and sterilized with an aqueous solution of 70% (v/v) ethanol according to previous methods (Yoon et al, Biotechnol Bioeng, 2002, 78:1-10; Yoon et al, Biomaterials, 2004, 25:5613-5620), and then placed in 24-well plates. A 100 μl of (3×10⁵ cells/100 μl) cell suspension was loaded onto the top surface of each pre-wetted scaffold and allowed to penetrate into the scaffold. The cells/scaffold constructs were then incubated at 37° C. under 5% CO₂ condition for 4 h for cell adherence. After cell adherence, the cells/scaffold constructs were transferred to a new 24-well plate in order to remove the lost cells at the bottom of the wells, and 1 ml of culture media was added in each new well containing the cells/scaffold construct. standard medium: DMEM containing 10% FBS (Hyclone, Logan, Utah), 1% nonessential amino acids and 100 U/ml penicillin/streptomycin (Gibco-BRL, Grand Island, N.Y.); and Culture media was changed every 2 days and culture plates were shaken during culture. At every indicated time interval, cells/scaffold constructs were collected for further experimental analysis.

### Example 8

### Cell Adherence and Viability Test in PLGA-ALN-3D and PLGA-3D Scaffold

For cell adherence tests, 4 h after cells adhere to the PLGA-ALN or PLGA scaffolds, cells/scaffold constructs were rinsed and removed from the 24-well plates. The number of unattached viable cells inside the wells were counted and compared with the control (24-well plate seeded cells without any scaffold) in order to get the number of viable cells attached to each scaffolds within the first 4 h. The CellTiter 96® AQueous One Solution Cell Proliferation Assay (Promega, Madison, Wis.) was used to count cell numbers, which is a colorimetric method for determining the number of viable cells in culture (Relic et al, J Immunol, 2001, 166:2775-2782). Briefly, the mitochondria activities of the hADSC cultured on wells were detected by the conversion of 3-(4,5-dimethylthiazol-2-yl)-2,5-diphenyltetrazolium bromide (MTS) to formazan as previously described (Relic et al, J Immunol, 2001, 166:2775-2782; Ma et al, Biomaterials, 2007, 28:1620-1628; Magne et al, J Bone Miner Res, 2003, 18:1430-1442), and the quantity of formazan product released into the medium, which is directly proportional to the number of living cells in culture, can be measured by absorbance at 490 nm (Relic et al, J Immunol, 2001, 166:2775-2782). At the indicated time interval, freshly prepared MTS reaction mixture diluted in standard medium at 1:5 (MTS:medium) volume ratio were added to the wells containing the cells and then incubated at 37° C. under 5% CO₂ for an additional 4 h. After the additional incubation, 100 μl of the converted MTS released into medium from each well was transferred to 96-well plates and the absorbance at 490 nm was recorded with a microplate reader (PathTech) using KC junior software. Cell adherence of hADSCs was calculated using the following formula:

Cell adherence (%)=[1−(Cell number unattached to scaffold/Control cell number inside wells)]×100%

The PLGA-ALN-3D showed the 80% of hADSCs were adhered on the scaffolds, which is significantly similar to PLGA-3D scaffolds (FIG. 1e).

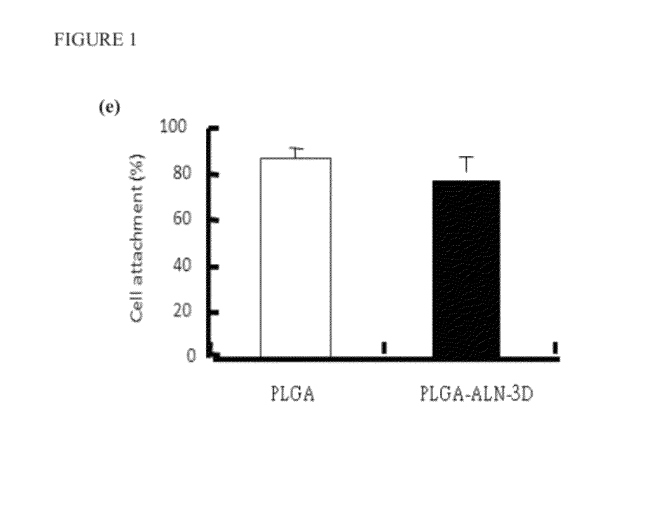

For cell viability tests, after the cells attached to the scaffold, the cells/scaffold constructs were transferred to a new culture plate and cultured in standard medium for an additional 1, 3, and 5 days at 37° C. under 5% CO₂. At every indicated time interval, freshly prepared MTS reaction mixture diluted in standard medium at 1:5 (MTS:medium) volume ratio were added to the wells, and the viable cell numbers within the constructs were assessed. The MTS assay results showed the PLGA-ALN-M or PLGA-ALN-3D treated hADSCs shows no adverse toxic effect at 1 and 3 days (FIG. 2).

### Example 9

### Osteogenic Differentiation

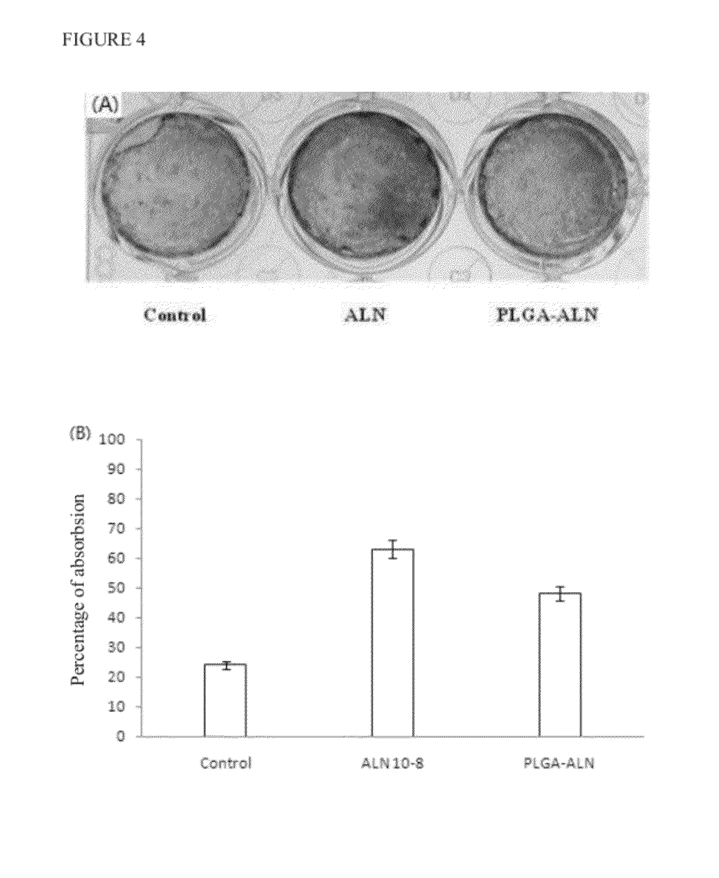

The ADSCs are seeded in PLGA-ALN-3D constructs at 10⁵ cells/well density followed by incubation for 12 hours, and add the conditioned medium (DMEM supplemented with 10% FBS, 100 units/ml penicillin and 100 g/ml streptomycin) and kept in incubator at 37° C., 5%, CO₂, for cultured for 7 days. After 7 days the culture medium was changed into osteoinduction medium and change every 2-3 days, after 14 days the cells are fixed by using 4% of the paraformaldehyde and tested the osteogenesis using Alizarin red S staining. The Alizarin red S staining showed the higher mineralization after 7 and 14 days in PLGA-ALN-M treated hADSCs cultures compared to the non-treated control cultures (FIG. 4).

### Example 10

### Alizarin red S Staining

Alizarin red S staining was used to determine the level of ECM (extra-cellular matrix) calcification 3 weeks after osteogenic induction. Cells were fixed with 4% paraformaldehyde at room temperature for 10 min. After washing once with ddH₂O, 1 ml alizarin red S solution (1% in ddH₂O, pH 4.2) was added to each well in the 12-well plate. The staining solution was removed 10 min later, and each well was washed with H₂O for 4-5 times. The fixed and stained plates were then air-dried at room temperature. The amount of mineralization was determined by dissolving the cell-bound alizarin red S in 10% acetic acid, and quantified spectrophotometrically at 415 nm.

### Example 11

### Osteogenic Differentiation of hADSCs

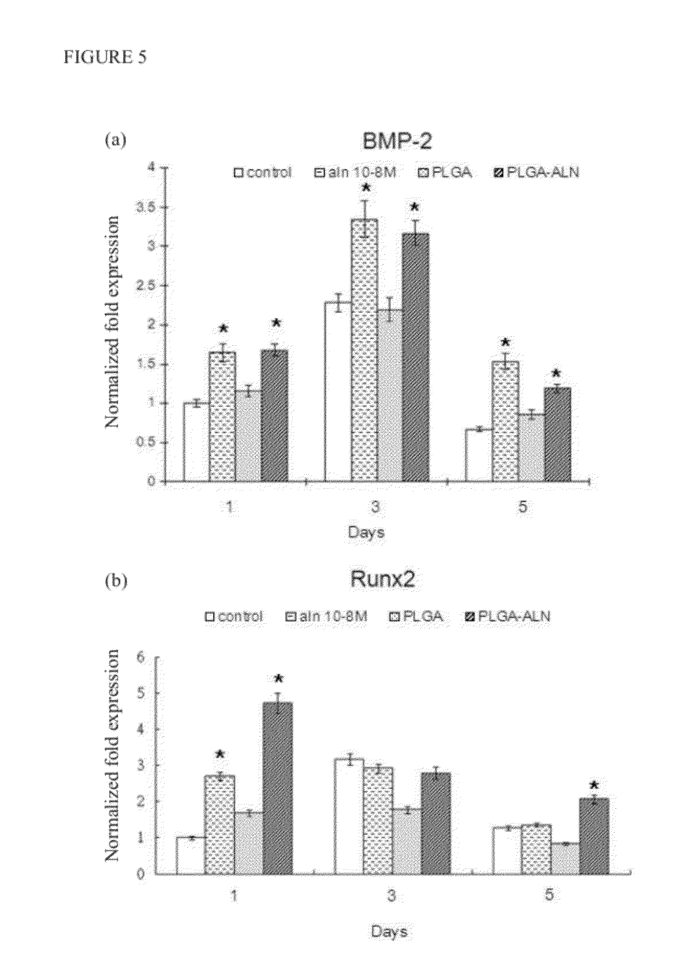

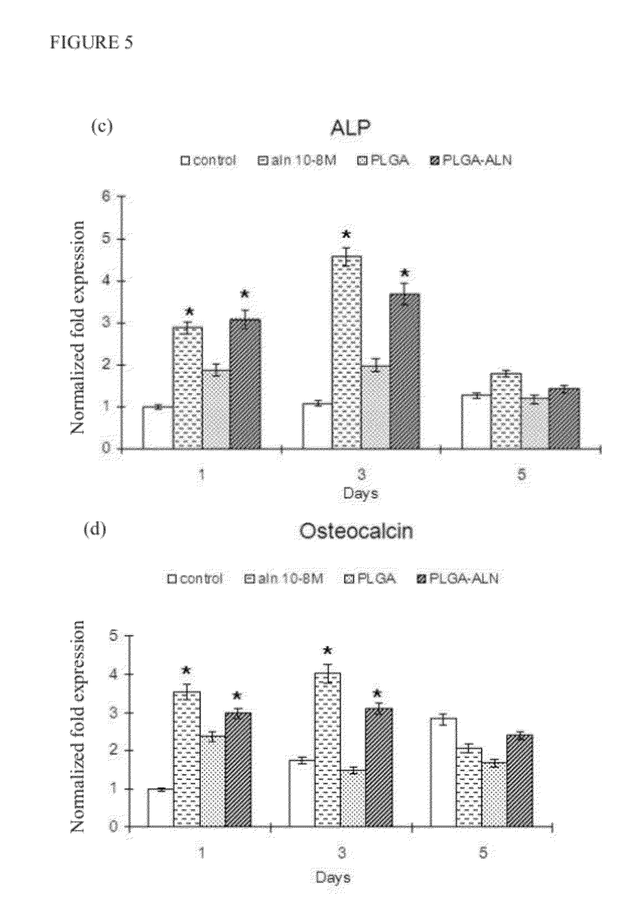

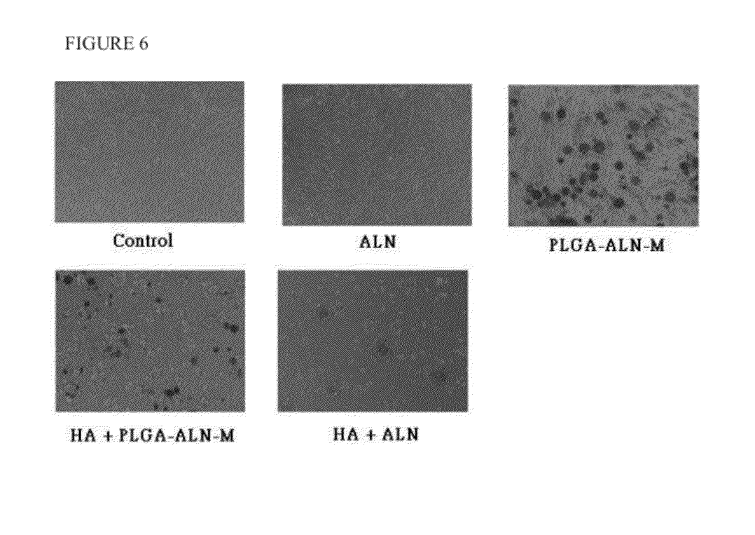

To evaluate osteogenic differentiation of hADSCs, mRNA expressions of osteogenic marker genes (Osteocalcin, Alkalinephosphatase, Runx2 and BMP-2) from cells cultured on scaffolds are examined by using real time PCR. The mRNA expressions of osteogenic marker genes osteocalcin, BMP-2, Alkalinephosphatase (ALP), and Runx2, were significantly increased (p>0.05) in 1, 3 and 5 days of PLGA-ALN-M treatment in hADSCs cultures in comparison with the control culture (FIG. 5).

### Example 12

### Chondrogenic Differentiation

The ADSCs are seeded in HA pre-coated trans-well fitted 24 well plate at 10⁵ cells/well density followed by incubation for 2 hours standing to form three-dimensional high-density micromass and 20 μL of PLGA-ALN-M (100 mg/mL) were treated through the trans-well, Add the conditioned medium (DMEM/10% FBS, 50 nM ascorbate-2-phosphate, 1% antibiotic/antimycotic), and kept in incubator at 37° C., 5%, CO2, for cultured for 14 days. After 7 days the trans-well with PLGA-ALN-M was removed and the culture medium was changed every 2-3 days.

### Example 13

### RNA Isolation and Real-Time Polymerase Chain Reaction (real-time PCR)

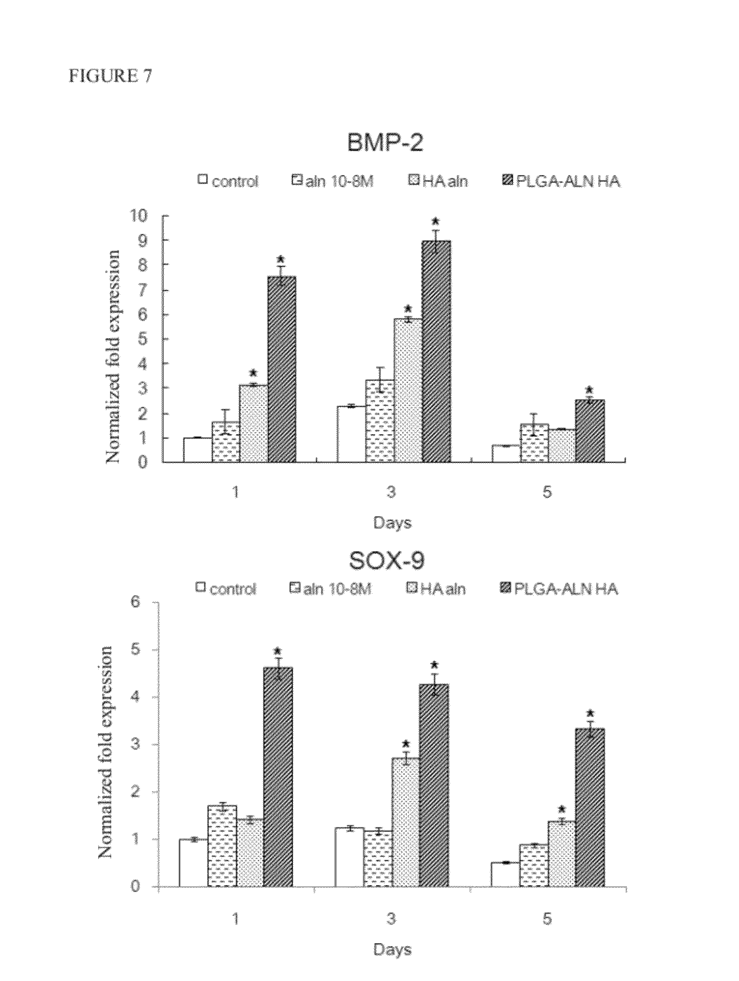

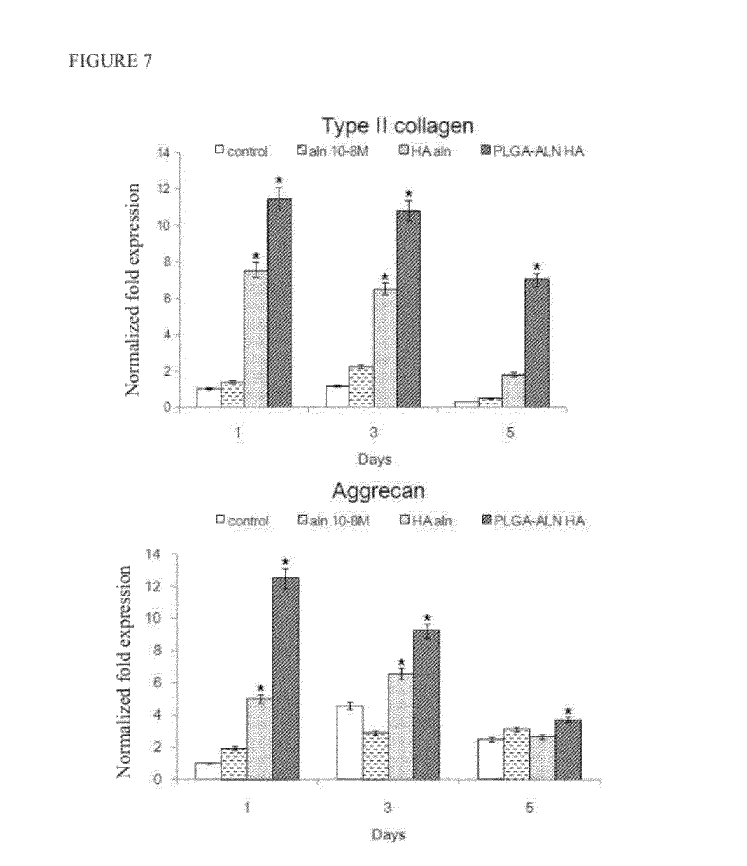

At indicated time intervals, cells were collected from cells/scaffold constructs. TRIzol (Gibco BRL, Rockville, Md.) was used to extract the total RNA from these cells by following manufacturer instructions. Briefly, 0.5-1 μg of total RNA per 20 μl of reaction volume were reverse transcribed into cDNA using the SuperScript First-Strand Synthesis System (Invitrogen). Real-time PCR reactions were performed and monitored using the iQ™ SYBR Green® supermix (Bio-Rad Laboratories Inc, Hercules, Calif.) and quantitative real-time PCR detection system (Bio-Rad Laboratories Inc, Hercules, Calif.). The cDNA samples (2 μl for total volume of 25 μl per reaction) were analyzed for gene of interest and the reference gene glyceraldehyde-3-phosphate-dehydrogenase (GAPDH). The expression level of each target gene was then calculated as 2-^{ΔΔCt}, as previously described (Livak and Schmittgen, Methods, 2001, 25(4):402-408). Four readings of each experimental sample were performed for each gene of interest, and experiments were repeated at least three times. The mRNA expressions of osteogenic marker genes osteocalcin, BMP-2, Alkalinephosphatase (ALP), and Runx2, were significantly increased (p>0.05) in 1, 3 and 5 days of PLGA-ALN-M treatment in hADSCs cultures in comparison with the control culture (FIG. 5). The mRNA expressions of chondrogenic marker genes such as BMP-2, SOX-9, collagen type II, and Aggrecan for chondrogenesis were significantly increased (p>0.05) in 1, 3, and 5 days on PLGA-ALN microspheres treated hADSCs cultured under HA microenvironment in comparison with control culture (FIG. 7).

### Example 14

### Animals and Surgery

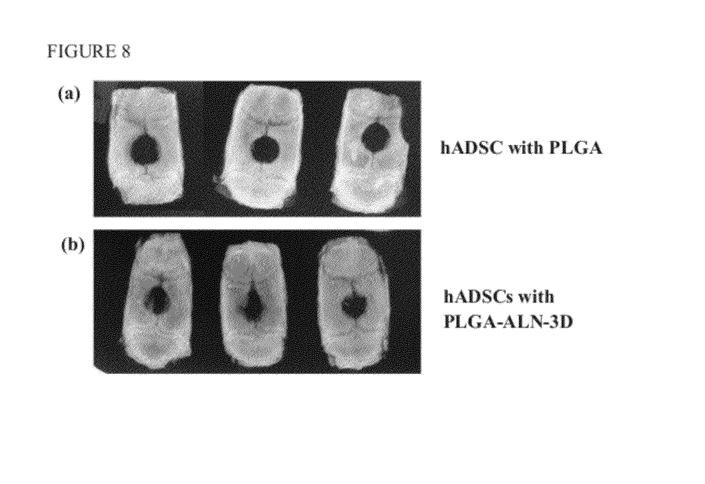

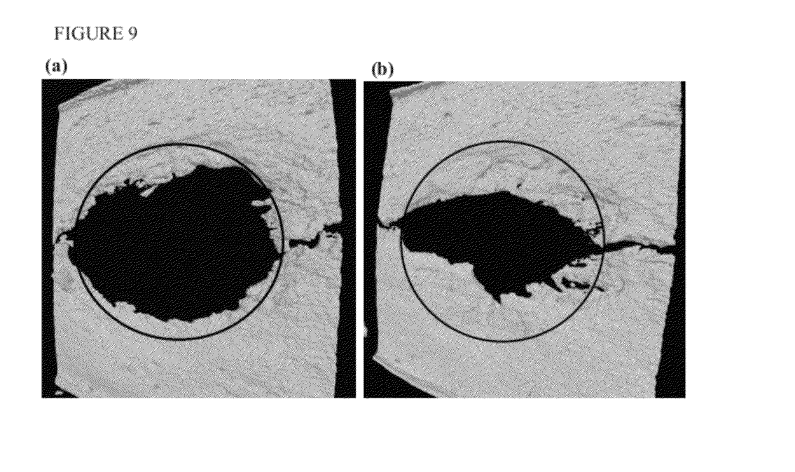

All animal experiments were performed in accordance with Kaohsiung Medical University Animal Care and Use Committee guidelines (IRB). Eighteen 8-10-week-old male Sprague Dawley rats (250-300 g) were housed in a light- and temperature-controlled environment and given food and water. Rats were anaesthetized with a combination of ketamine (75 mg/kg) and xylazine (10 mg/kg), administered intra-peritoneally. The dorsal part of the cranium was shaved, aseptically prepared for surgery, and a sagittal incision of approximately 20 mm opened over the scalp of the animal. The periosteum was removed and a full-thickness calvarial bone defect 5 mm in diameter was created using a slow speed dental drill without irrigation to heat damage the host bone on the rims and without damaging the dura. Bone defects were randomly implanted with hADSCs seeded PLGA-ALN-3D scaffolds or hADSCs seeded PLGA-3D scaffolds or left empty (n=6). Incisions were sutured and animals were allowed to recover for 8 weeks of post-surgery, after which they were sacrificed by CO₂ inhalation. To collect the implants, the skin was dissected, and the defect sites were removed along with surrounding bone. The specimens were fixed and prepared for μCT analysis and histology analysis. The radiographic images showed that the PLGA-ALN-3D constructs showed the better bone in growth in defect site of rat calvaria eight weeks after implantation (FIG. 8). The micro CT observation showed that the bone formation in rat calvarial defect model treated with PLGA-ALN-3D contracts showed better effect after eight weeks (FIG. 9).

### Example 15

### Histological and Immunochemical Analysis

To assess cell morphology and the presence of cartilage-specific matrix proteins, cells/scaffold constructs were fixed overnight in 4% paraformaldehyde in PBS (pH 7.4) at 4° C. and transferred to 70% ethanol until processing. Constructs were embedded in paraffin, and cut into 5 μm. For hitolocgical analysis, sections were stained with alcian blue for the presence of cartilage glycosaminglycan depositions. For immunohistochemistry, sections were also labeled with specific primary antibodies for collagen type II (dilution 1/100; Chemicon) followed FITC anti-mouse secondary antibodies (dilution 1/200; molecular probe). For negative control experiments, the primary antibodies were omitted. The sections were counterstained with 4′,6-Diamidino-2-phenylindole (DAPI) (dilution 1/500; Sigma) to identify cellular nuclei that reflected the cell number.

### Example 16

### Statistical Analysis

Three independent cultures for biochemical analysis were tested. Each experiment was repeated at least three times, and data (expressed as mean±SEM) from a representative experiment are shown. Statistical significance was evaluated by one-way analysis of variance (ANOVA), and multiple comparisons were performed by Scheffe's method. A p<0.05 was considered significant.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A short term controlled release composition which comprises poly(lactic-co-glycolic acid) (PLGA) cross-linked alendronate (ALN), wherein the released alendronate concentration is in the range of 5×10⁻⁷ M to 5×10⁻⁸ M.

2. The short term controlled release composition of claim 1, wherein the PLGA-ALN is constructed into 3D scaffolds (PLGA-ALN-3D) or microspheres (PLGA-ALN-M).

3. The short term controlled release composition of claim 2, wherein the PLGA-ALN-3D scaffolds have pores size of 150-300 μm and average porosity of 85%.

4. The short term controlled release composition of claim 2, wherein the PLGA-ALN-M is 50-100 μm in diameter.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)
